(19) United States
(12) Patent Application Publication
(10) Pub. No.: US 2009/0061000 A1
(43) Pub. Date: Mar. 5, 2009

(54) PHARMACEUTICAL FORMULATION USE 030

(75) Inventors: Bertil Sven Inge Abrahamsson, Molndal (SE); Susanna Johanna Abrahmsen Alami, Lund (SE); Hakan Lars Bagger-Jorgensen, Lund (SE); Marie Christine Sindeby Cullberg, Molndal (SE); Lars Johan Pontus de Verdier Hjartstam, Molndal (SE); Susanne Anette Nilsson, Lund (SE)

Correspondence Address: MORGAN LEWIS & BOCKIUS LLP, 1111 PENNSYLVANIA AVENUE NW, WASHINGTON, DC, 20004, US

(73) Assignee: AstraZeneca AB, Sodertalje (SE)

(21) Appl. No.: 12/200,549

(22) Filed: Aug. 28, 2008

Related U.S. Application Data
(60) Provisional application No. 60/969,188, filed on Aug. 31, 2007.

(51) Int. Cl. A61K 9/10; A61K 38/05; A61P 9/00

(52) U.S. Cl. 424/488; 514/19; 424/484

(57) ABSTRACT

An extended release pharmaceutical formulation comprising, as active ingredient, the compound Ph(3-Cl)(5-OCHF₂)—(R)CH(OH)C(O)—(S)Aze-Pab(OMe) or a pharmaceutically acceptable salt thereof (such as a sulfonic acid salt, such as the benzenesulfonic acid (besylate) salt); and a pharmaceutically acceptable diluent or carrier; for use in providing a therapeutic anti-thrombotic effect whilst limiting drug-drug interactions with other concomitantly dosed drug/s, particularly those which are metabolised by CYP-450 enzymes.

This application claims the benefit under 35 U.S.C. §119(e) of Application No. 60/969,188 (US) filed on 31 Aug. 2007.

This invention relates to certain extended release pharmaceutical formulations, the manufacture of such formulations and to their use in the treatment or prevention of thrombosis, in particular of systemic thromboembolism in patients with non-valvular atrial fibrillation and of venous thromboembolism.

International Patent Application No. WO 02/44145 discloses a number of compounds that are, or are metabolised to compounds which are, competitive inhibitors of trypsin-like proteases, such as thrombin. The following compound is amongst those that are specifically disclosed: Ph(3-Cl)(5-OCHF₂)—(R)CH(OH)C(O)—(S)Aze-Pab(OMe):

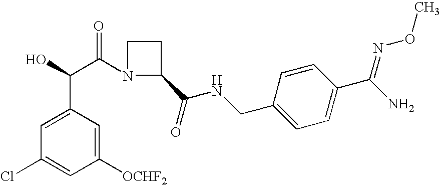

which compound is referred to hereinafter as Compound A.

Compound A is metabolised following oral and/or parenteral administration to a mammal and forms the corresponding free amidine compound, which latter compound has been found to inhibit thrombin. Thus, Compound A is metabolized to Ph(3-Cl)(5-OCHF₂)—(R)CH(OH)C(O)—(S)Aze-Pab (which compound is referred to hereinafter as Compound C) via a prodrug intermediate Ph(3-Cl)(5-OCHF₂)—(R)CH(OH)C(O)—(S)Aze-Pab(OH) (which compound is referred to hereinafter as Compound B). Processes for the synthesis of Compounds A, B and C are described in WO 02/44145.

As mentioned in WO 02/44145, Compound A may be dosed to patients who are also receiving other drugs, such as, for example, acetylsalicylic acid. Other possible combinations of drugs which a patient may receive include Compound A and, for example, digoxin. Further possible combinations of drugs which a patient may receive include Compound A and, for example, any one or more of the following drug/s; metformin, amiodarone, furosemide, metoprolol, amlodipine, verapamil, enalapril, losartan and/or simvastatin. A patient may also receive any other drug/s in the same class as any of the above-mentioned drug/s, such as another statin (such as atorvastatin or rosuvastatin) or another anti-platelet agent (such as, for example, clopidogrel). When combinations of drugs are administered there exists the possibility for drug-drug interactions. Such drug-drug interactions may arise from many factors, including metabolism or factors related to absorption, distribution and excretion. This may lead to altered drug exposure of potential importance for clinical efficacy and safety. It is therefore important to determine whether a particular drug will exhibit interactions with other drugs that a particular patient may be receiving, and if so, minimise any clinically undesirable interactions.

The metabolism of Compound A to Compound C via Compound B is mediated by cytochrome (CYP) P450 enzymes, including isoenzymes 2C9, 2C19 and 3A. Thus, there is a potential for pharmacokinetic interactions with other concomitantly used drugs that are metabolised by such P450 enzymes (such as CYP 450 3A) such as midazolam. Other such 3A(4)-substrates that may be commonly used include simvastatin, atorvastatin, amlodipine, diltiazem, prednisolone, verapamil and ketoconazole. Some compounds mediated by cytochrome (CYP) P450 enzymes may also be P-glycoprotein inhibitors, for example verapamil. 2C9-substrates commonly used may include losartan and glibenclamide.

Compound A can be formulated in certain formulations, for example modified release formulations (see WO 03/000293 and WO 03/101424) and immediate release formulations (see WO 03/101423), relevant sections from which are incorporated herein by reference.

Modified release dosage forms have increasingly become a method of delivering certain drugs to patients, particularly via the oral route. Such forms may, for example, provide for release of drug over an extended period of time.

Pharmaceutical formulations for administration of Compound A which provide different in-vivo plasma concentration versus time profiles, e.g. peak levels, may differ in the potential for Compound A to influence the pharmacokinetics of other drugs. However, it is not readily predictable whether a particular formulation for a particular drug will lead to an increase or decrease in the potential for drug-drug interactions with other, particular drugs.

According to a first aspect of the invention, there is provided an extended release pharmaceutical formulation comprising (as active ingredient), the compound Ph(3-Cl)(5-OCHF₂)—(R)CH(OH)C(O)—(S)Aze-Pab(OMe) or a pharmaceutically acceptable salt thereof (such as a sulfonic acid salt, such as the benzenesulfonic acid (besylate) salt); and a pharmaceutically acceptable diluent or carrier; for use in limiting drug-drug interactions whilst still providing a therapeutic anti-thrombotic effect.

In particular, the use of an extended release pharmaceutical formulation according to the invention limits the effect that Compound A, B or C has on other concomitantly dosed drugs which are metabolised by CYP-P450 enzymes, more specifically isoenzymes 3A, 2C9 and 2C19, in particular isoenzyme 3A.

In particular, the use of an extended release pharmaceutical formulation according to the invention limits the effect that Compound A, B or C has on other concomitantly dosed drugs which are absorbed, distributed or excreted via the same transporter protein as Compound A, B or C.

By “limits the effect that Compound A, B or C has on other concomitantly dosed drugs” we include a clinically insignificant effect on the exposure or plasma profile of the concomitantly dosed drug/s when Compound A, B or C are co-administered.

In another embodiment, the use of an extended release pharmaceutical formulation according to the invention delivers a peak plasma concentration of Compound A that is at least 2 times lower (particularly at least 3 times lower, and especially at least 4 times lower) than the plasma concentration of Compound A when administered using an immediate release formulation at the same level.

In another embodiment, the use of an extended release pharmaceutical formulation according to the invention is provided when the other concomitantly dosed drug or drugs is/are selected from any one of the following . . .

(i) a statin, such as simvastatin, pravastatin, atorvastatin or rosuvastatin;

(ii) amlodipine;

(iii) diltiazem;

(iv) prednisolone;

(v) verapamil;

(vi) losartan;

(vii) glibenclamide.

Compound A, or a pharmaceutically acceptable salt thereof (such as sulfonic acid salts, such as the benzenesulfonic acid (besylate) salt), may be in the form of a solvate, a hydrate, a mixed solvate/hydrate or, preferably, an ansolvate, such as an anhydrate. Solvates may be of one or more organic solvents, such as lower (for example C₁₋₄) alkyl alcohols (for example methanol, ethanol or iso-propanol), ketones (such as acetone), esters (such as ethyl acetate) or mixtures thereof.

The term “extended release” pharmaceutical composition will be well understood by the skilled person to include any composition/formulation in which the rate of release of drug is altered, i.e. extended, by galenic manipulations.

The invention also covers the use of the extended release formulations disclosed herein in the manufacture of a medicament for limiting the drug-drug interactions disclosed herein.

In the present case, extended release may be provided for by way of an appropriate pharmaceutically-acceptable carrier, and/or other means, which carrier or means (as appropriate) gives rise to an alteration of the rate of release of active ingredient. Thus, the term will be understood by those skilled in the art to include compositions which are adapted (for example as described herein) to provide for a “sustained”, a “prolonged” or an “extended” release of drug (in which drug is released at a sufficiently retarded rate to produce a therapeutic response over a required period of time).

More particular compositions of the invention may be adapted (for example as described herein) to provide a sufficient dose of drug over the dosing interval (irrespective of the number of doses per unit time) to produce a desired therapeutic effect. Release may be uniform and/or constant over an extended period of time, or otherwise.

Compositions of the invention may, for example, be in the form of the following, all of which are well known to those skilled in the art:

Formulations comprising dispersions or solid solutions of active compound in a matrix, which may be in the form of a wax, gum or fat, or, particularly, in the form of a polymer, in which drug release takes place by way of gradual surface erosion of the tablet and/or diffusion. Examples include gel matrix formulations, for example comprising HPMC.

Systems in which drug is released by diffusion through membranes, including multilayer systems. Examples include coated pellets, tablets or capsules. Further examples include multiple unit or multiparticulate systems, which may be in the form of microparticles, microspheres or pellets comprising drug (which multiple units/multiparticulates may provide for gradual emptying of the formulation containing drug from the stomach into the duodenum and further through the small and large intestine while releasing drug at a pre-determined rate).

Formulations using other extended release principles such as, for example, so-called “pendent” devices, in which drug is attached to an ion exchange resin, which provides for gradual release of drug by way of influence of other ions present in the gastrointestinal tract, for example, the acid environment of the stomach. Other such extended release principles include devices in which release rate of drug is controlled by way of its chemical potential (for example the Osmotic Pump) and silastic controlled release depots, which release drug as a function of diffusion of water and/or gastrointestinal fluids into the device via an entry/exit port, resulting in dissolution and subsequent release of drug.

The above principles are discussed at length in prior art references including Pharmaceutisch Weekblad Scientific Edition, 6, 57 (1984); Medical Applications of Controlled Release, Vol II, eds. Langer and Wise (1984) Bocaraton, Fla., at pages 1 to 34; Industrial Aspects of Pharmaceuticals, ed. Sandel, Swedish Pharmaceutical Press (1993) at pages 93 to 104; and pages 191 to 211 of “Pharmaceutics: The Science of Dosage Form Design”, ed. M. E. Aulton (1988) (Churchill Livingstone); as well as the references cited in the above-mentioned documents, the disclosures in all of which documents are hereby incorporated by reference. Suitable extended release formulations may thus be prepared in accordance with standard techniques in pharmacy, as described herein or in the above-mentioned documents, and/or which are well known.

The active ingredient is generally provided together with a pharmaceutically acceptable carrier. In particular, the compositions are presented in the form of active ingredient in a polymer matrix or pellet.

In this respect, particular compositions of the invention are provided for oral administration in the form of a so-called “swelling” modified-release system, or a “gelling matrix” modified-release system, in which active ingredient is provided together with a polymer that swells in an aqueous medium (that is a “hydrophilic gelling component”). The term “aqueous medium” is to be understood in this context to include water, and liquids which are, or which approximate to, those present in the gastrointestinal tract of a mammal. Such polymer systems typically comprise hydrophilic macromolecular structures, which in a dry form may be in a glassy, or at least partially crystalline, state, and which swell when contacted with aqueous media. Extended release of drug is thus effected by one or more of the following processes: transport of solvent into the polymer matrix, swelling of the polymer, diffusion of drug through the swollen polymer and/or erosion of the polymer, one or more of which may serve to release drug slowly from the polymer matrix into an aqueous medium.

Thus, suitable polymeric materials (acting as carriers), which may be used as the hydrophilic gelling component of a gelling matrix modified-release composition include those with a molecular weight of above 5000 g/mol, and which either:
(a) are at least sparingly soluble in; or
(b) swell when placed in contact with,
   aqueous media (as defined hereinbefore), so enabling release of drug from the carrier.

Suitable gelling matrix polymers, which may be synthetic or natural, thus include polysaccharides, such as maltodextrin, xanthan, iota-carrageenan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethylmethacrylate)); carboxypolymethylene (for example Carbopol™); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; poly(ethylene oxide) (PEO); and cellulose ethers, such as hydroxymethylcellulose (HMC), hydroxyethylcellulose (HEC), hydroxypropylcellulose (HPC), methylcellulose (MC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na CMC); as well as copolymers and/or (simple) mixtures of any of the above polymers. Certain of the above-mentioned polymers may further be crosslinked by way of standard techniques.

In a further aspect the invention provides use of an extended release formulation which comprises one or more polymers in a gelling matrix, particularly comprising hydroxy propyl methyl cellulose (HPMC). The HPMC may be one or a mixture of two or more HPMCs of different viscosities or molecular weights. In addition to HPMC, the formulation may also comprise a polymer with pH dependent solubility such as polymethacrylic acid and/or methacrylic acid copolymer/s. Additionally, the formulation may comprise one or more further components selected from the group comprising microcrystalline cellulose, a lubricant (such as sodium stearyl fumarate) or mannitol.

Suitable HPMC polymers also include those that produce 2% w/w solutions of polymer in water with viscosities, as measured by standard techniques, such as those described generally in the United States Pharmacopeia XXIV (USP XXIV/NF19) at page 2002 et seq, as well as, specifically, at pages 843 and 844 (the relevant disclosures in which document are hereby incorporated by reference), of between 3 and 150,000 cps (at 20° C.), such as between 10 and 120,000 cps, preferably between 30 and 50,000 cps and more preferably between 50 and 15,000 cps. Mixtures of HPMC polymers with different viscosities within these ranges may be employed, in order, for example, to produce HPMC mixtures which produce solutions as mentioned above with “average” viscosities (i.e. a viscosity for the mixture) within the above-mentioned preferred ranges. Similarly, mixtures of HPMC polymers (with viscosities and/or “average” viscosities within these ranges) with other above-mentioned polymers may be employed. Suitable HPMC polymers include those fulfilling the United States Pharmacopeia standard substitution types 2208, 2906, 2910 and 1828 (see USP XXIV/NF19 for further details). Suitable HPMC polymers thus include those sold under the trademark METHOCEL™ (Dow Chemical Corporation) or the trademark METOLOSE™ (Shin-Etsu).

The choice of polymer will be determined by the nature of the active ingredient/drug that is employed as well as the desired rate of release. In particular, it will be appreciated by the skilled person, for example in the case of HPMC, that a higher molecular weight will, in general, provide a slower rate of release of drug from the composition. Furthermore, in the case of HPMC, different degrees of substitution of methoxyl groups and hydroxypropoxyl groups will give rise to changes in the rate of release of drug from the composition. In this respect, and as stated above, it may be desirable to provide compositions of the invention in the form of gelling matrix systems in which the polymer carrier is provided by way of a blend of two or more polymers of, for example, different molecular weights, for example as described hereinafter, in order to produce a particular required or desired release profile.

When in the form of gelling matrix systems, we have also found that rate of release of drug from compositions used in the invention may be further controlled by way of controlling the drug:polymer ratio within, and the surface area:volume ratio of, individual compositions (for example tablets) comprising drug and polymer carrier system.

In a further aspect, compositions of the invention are provided for oral administration in the form of pellets or multiple unit systems. Such multiple unit or multiparticulate systems can be produced by a number of processes, including spray layering or spray crystallization in a fluidised bed, melt spheronization, extrusion/spheronization, powder layering and rotor granulation. In the case of pellets produced by the spray layering technique, the active ingredient is dispersed in an aqueous medium and sprayed onto inert cores in a fluid bed equipment. The inert cores can, for example, be made of micro-crystalline cellulose. The pellets thus formed may then be coated by spraying a solution or a dispersion of one or more polymers on top of the substance layer in order to control the release of active substance. The polymer coating (or coatings if there are a number of polymer layers) thus obtained may comprise one or more polymers which may, for example, possess different physicochemical properties such as solubility in aqueous media. The choice of polymers and ratio between the included polymers will be determined by the nature of the active ingredient/drug as well as the desired rate of release. Suitable coating polymers include ethyl cellulose (EC), hydroxypropyl cellulose (HPC) and pH dependent soluble polymers such as methacrylic acid copolymer.

Compositions used in the invention, whether in the form of a gelling matrix system or a multiple unit system or otherwise, may contain one or more further excipients to further modify drug release, to improve the physical and/or chemical properties of the final composition, and/or to facilitate the process of manufacture. Such excipients are conventional in the formulation of modified release compositions.

For example, compositions used in the invention may contain one or more of the following diluents: calcium phosphate (monocalcium phosphate, dicalcium phosphate and tricalcium phosphate), lactose, microcrystalline cellulose, mannitol, sorbitol, titanium dioxide, aluminium silicate and the like. Preferred diluents include microcrystalline cellulose and also mannitol.

Compositions used in the invention may contain one or more of the following lubricants: magnesium stearate, sodium stearyl fumarate and the like.

Compositions used in the invention may contain a glidant, such as a colloidal silica.

Compositions used in the invention may contain one or more of the following binders: polyvinylpyrrolidone, lactose, mannitol, microcrystalline cellulose, a polyethylene glycol (PEG), a methacrylic acid copolymer/s, a HPMC of a low molecular weight, a MC of a low molecular weight, a HPC of a low molecular weight and the like. Preferred binders include microcrystalline cellulose and HPC.

Compositions used in the invention may contain one or more of the following pH controlling agents: suitable polymers (such as polymethacrylic acid and/or methacrylic acid copolymer/s), organic acids (for example, citric acid and the like) or alkali metal (for example sodium) salts thereof, pharmaceutically acceptable salts (for example sodium, magnesium or calcium salts) of inorganic acids (such as carbonic acid or phosphoric acid), oxides of magnesium, as well as alkali, and alkaline earth metal (for example sodium, calcium, potassium and the like) sulphates, metabisulphates, propionates and sorbates.

Other further excipients may include colourants, flavourings, solubilising agents, surfactants, coating agents, preservatives and plasticizers etc.

Combinations of the above-stated further excipients may be employed.

Suitable tablet coatings may comprise HPMC (Hypromellose, e.g. 6 cPs); PEG (macrogols); titanium dioxide; colour iron oxide yellow or red and water (q.s.). Typically, a suitable coating comprises up to 5 wt.% of a formulation.

It will be appreciated that some of the above mentioned further excipients, which may be present in the final composition used in the invention, may have more than one of the above-stated functions. Moreover, further excipients mentioned above may also function as part of a hydrophilic gelling component in a gelling matrix system.

The total amount of further excipients (not including, in the case of gelling matrix systems, the principal polymer carrier(s)) that may be present in the composition used in the invention will depend upon the nature of the composition, as well as the nature, and amounts of, the other constituents of that composition, and may be an amount of up to 85%, for example between 0.1 to 75%, such as 0.2 to 65%, preferably 0.3 to 55%, more preferably 0.5 to 45% and especially 1 to 40%, such as 2 to 35% w/w. In any event, the choice, and amount, of excipient(s) may be determined routinely (that is without recourse to inventive input) by the skilled person.

In gelling matrix systems, the amount of polymer in the system should be enough to ensure that a sufficient dose of drug is provided over the dosing interval to produce the desired therapeutic effect. Thus, for a gelling matrix system, we prefer that it takes at least 4 hours (especially at least 6 hours) for 80% (especially 60%) of the initial drug content of the composition to be released to a patient after administration under the test conditions described hereinafter, and particularly over a period of between 8 and 24 hours. Most preferably at least 80% of the initial drug content of the composition is released at a time somewhere between 8 and 24 hours. Suitable amounts of polymer that may be included, which will depend upon inter alia the active ingredient that is employed in the composition, any excipients that may be present and the nature of the polymer that is employed, are in the range 5 to 99.5%, for example 10 to 95%, preferably 30 to 80% w/w. In any event, the choice, and amount, of polymer may be determined routinely by the skilled person.

The neutral gelling polymer may be used as a single, or a mixture of more than one, neutral erodable polymer(s) having gelling properties and having substantially pH-independent solubility. The neutral gelling polymer is, preferably, present in the formulation at a level of more that 10% but preferably more than 20% by weight. Additionally, charged polymers (such as, for example, iota-carrageenan or methacrylic acid copolymer/s) may also be present.

Particular additional excipients in such formulations include lubricants, such as sodium stearyl fumarate (for example, in a range of 0.1-2.5 wt. %, or 0.5-1.25 wt. % of the formulation). In one aspect the invention provides use of a non-injectable formulation of the invention comprising Compound A, or a pharmaceutically-acceptable salt (such as sulfonic acid salts, such as the benzenesulfonic acid (besylate) salt) thereof, an HPMC and a lubricant (such as sodium stearyl fumarate).

In a further aspect the formulation may comprise a mixture of 2 or more HPMCs of different viscosities (such as 10,000 cPs and 50 cPs). Suitable amounts of active ingredient in the compositions use in the invention, whether in the form of gelling matrix systems or otherwise, depend upon many factors, such as the nature of that ingredient (free base/salt etc.), the dose that is required, and the nature, and amounts, of other constituents of the composition. However, they may be in the range 0.5 to 80%, for example 1 to 75%, such as 3 to 70%, preferably 5 to 65%, more preferably 10 to 60% and especially 15 to 55% w/w. In any event, the amount of active ingredient to be included may be determined routinely by the skilled person.

A typical daily dose of a compound A, or a pharmaceutically acceptable salt thereof, is in the range 0.001 to 100 mg/kg body weight of free base (that is, in the case of a salt, excluding any weight resulting from the presence of a counter ion), irrespective of the number of individual doses that are administered during the course of that day. A particular daily dose is in the range 20-1,000 mg; 50-750 mg or 20-500 mg; especially 150-600 mg or 100-500 mg.

Compositions used in the invention such as those described hereinbefore may be made in accordance with well known techniques such as those described in the references mentioned hereinbefore. Compositions of the invention that are in the form of gelling matrix systems may be prepared by standard techniques, and using standard equipment, known to the skilled person, including wet or dry granulation, direct compression/compaction, drying, milling, mixing, tabletting and coating, as well as combinations of these processes, for example as described hereinafter.

Although compositions used in the invention are preferably adapted to be administered orally, their use is not limited to that mode of administration. Parenteral modified release compositions of the invention, which may include systems that are well known to those skilled in the art, such as those based upon poloxamers, biodegradable microspheres, liposomes, suspensions in oils and/or emulsions, may be prepared in accordance with standard techniques, for example as described by Leung et al in “Controlled Drug Delivery: Fundamentals and Applications” (Drugs and the Pharmaceutical Sciences; vol. 29), 2^{nd} edition, eds. Robinson and Lee, Dekker (1987) at Chapter 10, page 433, the disclosure in which document is hereby incorporated by reference.

The compositions used in the invention may be dosed once or more times daily (preferably once, but no more than twice, daily), irrespective of the number of individual units (formulations/compositions) that are administered as part of one “dose”.

According to a further aspect of the invention there is thus provided use of a formulation of the invention for use as a pharmaceutical.

In particular, compound A is metabolised following administration to form a potent inhibitor of thrombin as may be demonstrated in the tests described in inter alia international patent application No. PCT/SE01/02657, as well as international patent applications WO 02/14270, WO 01/87879 and WO 00/42059, the relevant disclosures in which documents are hereby incorporated by reference.

By “active ingredient” and “active (drug) substance” we mean the pharmaceutical agent (covering thrombin inhibitor and prodrugs thereof) present in the formulation. By “prodrug of a thrombin inhibitor”, we include compounds that are metabolised following administration and form a thrombin inhibitor, in an experimentally-detectable amount, following administration.

The formulations used in the invention are thus expected to be useful in those conditions where inhibition of thrombin is required, and/or conditions where anticoagulant therapy is indicated, including the following:

The treatment and/or prophylaxis of thrombosis and hypercoagulability in blood and/or tissues of animals including man. It is known that hypercoagulability may lead to thrombo-embolic diseases. Conditions associated with hypercoagulability and thrombo-embolic diseases which may be mentioned include inherited or acquired activated protein C resistance, such as the factor V-mutation (factor V Leiden), and inherited or acquired deficiencies in antithrombin III, protein C, protein S, heparin cofactor II. Other conditions known to be associated with hypercoagulability and thrombo-embolic disease include circulating antiphospholipid antibodies (Lupus anticoagulant), homocysteinemi, heparin induced thrombocytopenia and defects in fibrinolysis, as well as coagulation syndromes (for example disseminated intravascular coagulation (DIC)) and vascular injury in general (for example due to surgery).

The treatment of conditions where there is an undesirable excess of thrombin without signs of hypercoagulability, for example in neurodegenerative diseases such as Alzheimer's disease.

Particular disease states which may be mentioned include the therapeutic and/or prophylactic treatment of venous thrombosis (for example DVT) and pulmonary embolism, arterial thrombosis (e.g. in myocardial infarction, unstable angina, thrombosis-based stroke and peripheral arterial thrombosis), and systemic embolism usually from the atrium during atrial fibrillation (for example, non-valvular atrial fibrillation, paroxysmal AF, persistent AF or permanent AF) or from the left ventricle after transmural myocardial infarction, or caused by congestive heart failure; prophylaxis of re-occlusion (that is thrombosis) after thrombolysis, percutaneous trans-luminal angioplasty (PTA) and coronary bypass operations; the prevention of re-thrombosis after microsurgery and vascular surgery in general.

Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis; the therapeutic and/or prophylactic treatment of idiopathic and adult respiratory distress syndrome, pulmonary fibrosis following treatment with radiation or chemotherapy, septic shock, septicemia, inflammatory responses, which include, but are not limited to, edema, acute or chronic atherosclerosis such as coronary arterial disease and the formation of atherosclerotic plaques, cerebral arterial disease, cerebral infarction, cerebral thrombosis, cerebral embolism, peripheral arterial disease, ischaemia, angina (including unstable angina), reperfusion damage, restenosis after percutaneous trans-luminal angioplasty (PTA) and coronary artery bypass surgery.

The formulations used in the present invention may also comprise any antithrombotic agent(s) with a different mechanism of action to that of the compounds A, such as one or more of the following: the antiplatelet agents acetylsalicylic acid, ticlopidine and clopidogrel; thromboxane receptor and/or synthetase inhibitors; fibrinogen receptor antagonists; prostacyclin mimetics; phosphodiesterase inhibitors; ADP-receptor (P₂T) antagonists; and inhibitors of carboxypeptidase U (CPU).

Compounds which inhibit trypsin and/or thrombin may also be useful in the treatment of pancreatitis, including chronic pancreatitis and pancreatic pain.

The formulations used in the invention are thus indicated both in the therapeutic and/or prophylactic treatment of these conditions.

The formulations used in the invention are useful in the delivery of a compound A or a salt thereof to a patient. As compound A, and salts thereof, are useful in both the prophylaxis and the treatment of thrombosis, the formulations used in the invention are also useful in the treatment of such a disorder. When using compound A, and salts thereof, in such treatment, a suitable assay, such as, for example, Thrombin Time or Ecarin Clotting Time, may be used to monitor the anti-coagulation.

According to a further aspect of the invention, there is provided a method of treatment of thrombosis whilst limiting drug-drug interactions which method comprises administration of a therapeutically effective amount of a formulation used according to the invention to a person suffering from, or susceptible to, such a condition.

According to a further aspect of the invention, there is provided a method of treatment of chronic pancreatitis which method comprises administration of a therapeutically effective amount of a formulation according to the invention to a person suffering from, or susceptible to, such a condition. In a still further aspect the present invention provides use of a formulation used in the invention in the manufacture of a medicament for use in the treatment of thrombosis.

For the avoidance of doubt, by “treatment” we include the therapeutic treatment, as well as the prophylaxis, of a condition.

The compositions used in the invention have an advantage that they may provide an extended release of the compound A, or a pharmaceutically acceptable salt thereof, in order to obtain a more even and/or prolonged effect against thrombosis and may thus provide efficient dosing of active ingredient (particularly no more than once or twice daily) whilst limiting drug-drug interactions.

Compositions used in the invention may also have the advantage that they may be prepared using established pharmaceutical processing methods and employ materials that are approved for use in foods or pharmaceuticals or of like regulatory status.

### EXAMPLES

The invention is illustrated, but in no way limited, by the following Examples. Further features of the invention include a formulation as described in herein, in particular according to any of the Examples, and a product obtainable by following any of the Examples or processes described herein.

Further Examples may be prepared by analogous procedures to those described herein with the composition adjusted proportionately for different tablet strengths.

### Example 1-A

### Immediate Release Formulation

The composition and preparation of the immediate release formulation used in the following Example 1-C is described below.

| Components | Quantity (mg/tablet) |
|---|---|
| Compound A besylate | 164.8 |
| (corresponding to Compound A 125 mg) | |
| Cellulose, microcrystalline | 68.7 |
| Hypromellose (HPMC) | 8.2 |
| Macrogols | 2.1 |
| Mannitol | 13.7 |
| Hydroxy propyl cellulose | 11.0 |
| Sodium starch glycolate (Type A) | 13.7 |
| Sodium stearyl fumarate | 2.7 |
| Titanium dioxide | 2.1 |
| Water, purified^{a,b} | q.s. |

^{a}Water (purified) is used as granulating fluid during manufacture of the tablet core and is removed during granule drying.
^{b}Water (purified) is used as the solvent/carrier fluid during film coating and is removed during the coating process.

Compound A besylate tablets were manufactured using conventional mixing, wet granulation, drying, milling, blending, compression and film coating processes.

The granulating solution was prepared by dissolution of the binder, hydroxy propyl cellulose or povidone, in purified water. The drug substance, microcrystalline cellulose, mannitol, and sodium starch glycolate were mixed to produce a uniform distribution of the drug substance. The powder mix was granulated by adding the granulating solution while mixing, followed by additional wet mixing. The granulated wet mass was thereafter dried to produce a granulated mass with a suitable moisture content. The dried mass was milled through a suitable mill or sieved through a suitable screen in order to obtain a granulate of a suitable size. The lubricant, sodium stearyl fumarate,was charged through a sieve to the granulate, blended and the blend was compressed into tablet cores using conventional tabletting equipment. The coating liquid was prepared by dissolution of hypromellose and macrogols in purified water, followed by suspension of titanium dioxide into this solution.

### Example 1-B

### Extended Release Formulation

The composition and preparation of the extended release formulation used in the following Example 1-C is described below.

| | Quantity |
|---|---|
| Components | (mg/tablet) |
| Compound A besylate | 132 |
| (corresponding to Compound A 100 mg) | |
| Cellulose, microcrystalline | 60.0 |
| Hypromellose 50 mPas | 154 |
| Sodium stearyl fumarate | 7.0 |
| Ethanol, anhydrous (removed during the processing | q.s. |

Compound A tablets were manufactured using conventional mixing, wet granulation, drying, milling, blending, compression and film coating processes.

The powder mix was granulated by adding the granulation liquid (ethanol) whilst mixing, followed by additional wet mixing; if necessary, more ethanol being added.

The wet mass was dried in a hot air oven or a fluid bed dryer. The dried mass was then milled through a suitable mill or sieved through a suitable screen.

The granulate was mixed with microcrystalline cellulose and sodium stearyl fumarate, which was charged through a suitable sieve. The granulate was compressed into tablets using a tablet press equipped with convex punches.

### Example 1-C

### Comparison of Immediate (IR) and Extended Release (ER) Formulations

24 healthy male subjects aged between 20 and 43 years received the following dosing regimens in random order:
1. Single oral doses of tolbutamide 500 mg and midazolam 7.5 mg
2. Single oral doses of tolbutamide 500 mg and midazolam 7.5 mg together with a single oral dose of 500 mg of Compound A given as 4×125 mg IR tablets prepared as in Example 1-A.
3. Single oral doses of tolbutamide 500 mg and midazolam 7.5 mg together with a single oral dose of 500 mg of Compound A given as 5×100 mg ER tablets prepared as in Example 1-B.

Tolbutamide was administered as 1×500 mg commercially available IR tablet and midazolam as 1×7.5 mg commercially available IR tablet. The three treatments were separated by a wash-out period of 7 to 21 days.

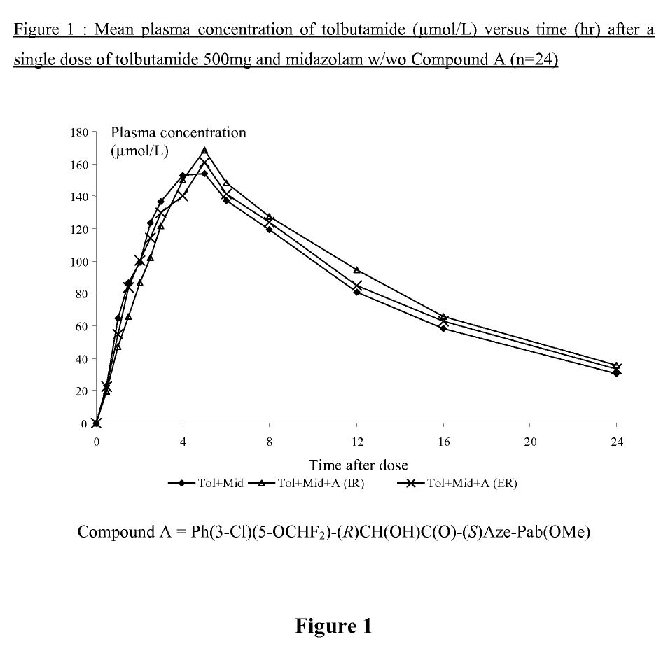

After administration, blood was collected and the plasma concentration of tolbutamide, midazolam and Compound A determined by High Performance Liquid Chromatography tandem Mass Spectrometry (HPLC-MS/MS) methods. The resulting plasma concentration vs. time curves are shown in FIGS. 1, 2 and 3 for Tolbutamide, midazolam and Compound A respectively.

FIG. 1 shows Mean plasma concentration of tolbutamide (μmol/L) versus time (hr) after a single dose of tolbutamide 500 mg and midazolam w/wo Compound A (n=24).

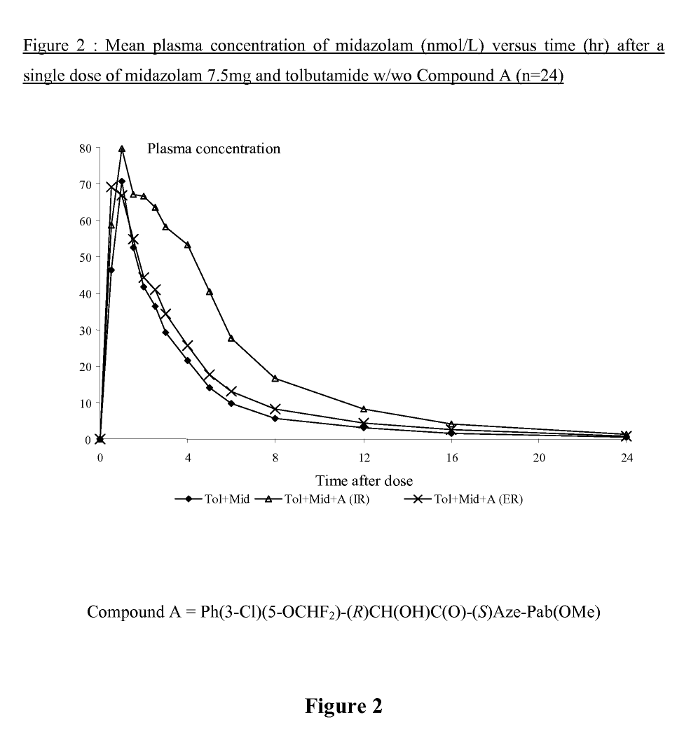

FIG. 2 shows Mean plasma concentration of midazolam (nmol/L) versus time (hr) after a single dose of midazolam 7.5 mg and tolbutamide w/wo Compound A (n=24).

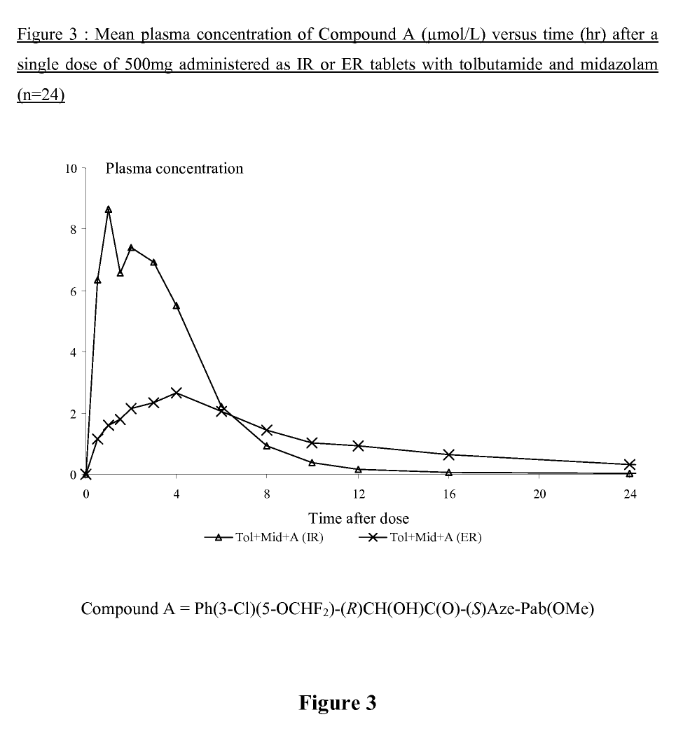

FIG. 3 shows Mean plasma concentration of Compound A (μmol/L) versus time (hr) after a single dose of 500 mg administered as IR or ER tablets with tolbutamide and midazolam (n=24).

The area under the plasma concentration versus time curves from zero to the last quantifiable plasma concentration (AUC₀₋ₜ) in FIGS. 1, 2 and 3 are shown in the table below.

Mean ± standard deviation of area under the plasma concentration
versus time curves of tolbutamide and midazolam

| | Tolbutamide | Midazolam |
|---|---|---|
| | AUC₀₋ₜ | AUC₀₋ₜ |
| Treatment | (μmol * h/L) | (μmol * h/L) |
| Tolbutamide + midazolam | 1944 ± 309 | 232 ± 81 |
| Tolbutamide + midazolam + | 2067 ± 324 | 450 ± 168 |
| Compound A besylate (IR) | | |
| Tolbutamide + midazolam + | 1997 ± 323 | 281 ± 111 |
| Compound A besylate (ER) | | |

Administration of Compound A as an ER formulation has less effect on CYP3A actvity compared to administration as an IR formulation, as judged by a minimal increase in midazolam AUC_{0-T} after co-administration with Compound A as an ER formulation and an approximately two-fold increase in AUC_{0-T} of midazolam after co-administration with compound A given as an IR formulation.

Administration of Compound A had no effect on CYP2C9 activity, as judged by no influence on the pharmacokinetics of tolbutamide after co-administration with Compound A.

In the above cocktail study the Compound A peak levels were >3.5-fold lower for ER (3.45 μmol/L) compared to IR (13.1 μmol/L) i.e. lowered by approximately 75%. Other extended release formulations which possess a suitable profile for limiting drug-drug interactions (compared to immediate release formulations) as described herein are as follows. Such formulations may also possess other desirable profiles such as, for example, robustness regarding effects when dosed with food (e.g. food may cause an increase in release rate resulting in higher plasma drug peak levels).

### Example 2

### Extended Release Tablets

| | |
|---|---|
| Compound A besylate | 198.0 mg |
| Hypromellose 50 mPas | 102.6 mg |
| Cellulose, microcrystalline | 18.0 mg |
| Methacrylic acid - methyl methacrylate copolymer (1:1) | 36.0 mg |
| Ethanol, anhydrous (removed during processing) | q.s. |
| Sodium stearyl fumarate | 5.4 mg |

Hypromellose 50 mPas, microcrystalline cellulose and Compound A besylate were blended for 3 minutes. The powder blend was granulated by adding the granulation liquid consisting of methacrylic acid—methyl methacrylate copolymer (1:1) in ethanol while mixing for approximately 5 minutes, followed by additional wet mixing. The wet mass was milled in a Quadro Comill.

The wet mass was dried in a hot air oven or a fluid bed drier and the dried mass was milled in a Fitz Mill. The granules were final mixed with sodium stearyl fumarate, which was charged through a suitable sieve. The granules were compressed into tablets using a tablet press equipped with convex punches.

### Example 3

### Extended Release Tablets

| | |
|---|---|
| Compound A besylate | 198.0 mg |
| Hypromellose 50 mPas | 129.6 mg |
| Cellulose, microcrystalline | 18.0 mg |
| Methacrylic acid - methyl methacrylate copolymer (1:1) | 9.0 mg |
| Ethanol, anhydrous (removed during processing) | q.s. |
| Sodium stearyl fumarate | 5.4 mg |

Hypromellose, microcrystalline cellulose and Compound A besylate were blended for 3 minutes. The powder blend was granulated by adding the granulation liquid consisting of methacrylic acid—methyl metacrylate copolymer (1:1) in ethanol while mixing for approximately 5 minutes, followed by additional wet mixing. The wet mass was milled in a Quadro Comill.

The wet mass was dried in a hot air oven or a fluid bed drier and the dried mass was milled in a Fitz Mill. The granules were final mixed with sodium stearyl fumarate, which was charged through a suitable sieve. The granules were compressed into tablets using a tablet press equipped with convex punches.

### Example 4

### Extended Release Capsules

| Compound A besylate | 198 | mg |
|---|---|---|
| Cellulose, microcrystalline | 36.7 | mg |
| Ethanol, anhydrous^{b} | q.s. | |
| Ethylcellulose | 48.8 | mg |
| Glyceryl monostearate 40-55 | 5.95 | mg |
| Hard gelatin capsules | Approx 130 | mg |
| Hydrochloric acid, concentrated^{a} | Approx 1 | mg |
| Hydroxypropyl cellulose | 28.6 | mg |
| Hypromellose | 22.0 | mg |
| Methacrylic acid-ethyl acrylate copolymer (1:1) | 119 | mg |
| dispersion 30 percent^{a} | | |
| Polysorbate 80 | 0.59 | mg |
| Triethyl citrate | 11.9 | mg |

| | |
|---|---|
| Water, purified^{b} | q.s. |

^{a}The amount expressed on dry basis.
^{b}Removed during processing

A suspension of Compound A was sprayed onto microcrystalline cellulose spheres in a fluidised bed with subsequent drying. The uncoated pellets were coated in a fluid bed using an ethanol based solution of ethylcellulose (EC) and hydroxypropylcellulose (HPC) and were subsequently dried.

The pellets coated with ethylcellulose (EC) and hydroxypropylcellulose (HPC) were further coated in a fluidised bed using a dispersion consisting of methacrylic acid-ethyl acrylate copolymer (1:1) dispersion 30 per cent, glycerol monostearate, triethyl citrate and polysorbate and were subsequently dried. The film-coated pellets were then filled into hard gelatin capsules.

### Example 5

### Extended Release Capsules

| Compound A | 150 | mg |
|---|---|---|
| Cellulose, microcrystalline | 56.3 | mg |
| Ethanol, anhydrous^{b} | q.s. | |
| Ethylcellulose | 17.0 | mg |
| Glyceryl monostearate 40-55 | 3.08 | mg |
| Hard gelatin capsules, | Approx 130 | mg |
| Hydroxypropyl cellulose | 20.7 | mg |
| Hypromellose | 12.9 | mg |
| Methacrylic acid-ethyl acrylate copolymer (1:1) | 61.7 | mg |
| dispersion 30 percent^{a} | | |
| Polysorbate 80 | 0.31 | mg |
| Triethyl citrate | 6.17 | mg |

| | |
|---|---|
| Water, purified^{b} | q.s. |

^{a}The amount expressed on dry basis.
^{b}Removed during processing

A suspension of Compound A was sprayed onto microcrystalline cellulose spheres in a fluidised bed with subsequent drying.

The uncoated pellets were coated in a fluid bed using a dispersion consisting of methacrylic acid-ethyl acrylate copolymer (1:1) dispersion 30 per cent, glycerol monostearate, triethyl citrate and polysorbate and were subsequently dried.

The pellets coated with methacrylic acid-ethyl acrylate copolymer (1:1) dispersion 30 per cent were further coated in a fluidised bed using an ethanol based solution of ethylcellulose (EC) and hydroxypropylcellulose (HPC) and were subsequently dried. The film-coated pellets were then filled into hard gelatin capsules.

### Example 6

### Extended Release Tablet

| | |
|---|---|
| Compound A besylate | 198.0 mg |
| Hypromellose 50 mPas | 113.4 mg |
| Cellulose, microcrystalline | 18.0 mg |
| Methacrylic acid - methyl methacrylate copolymer (1:1) | 25.2 mg |
| Ethanol, anhydrous (removed during processing) | q.s. |
| Sodium stearyl fumarate | 3.6 mg |

Hypromellose 50 mPas, microcrystalline cellulose and Compound A besylate were blended for 3 minutes. The powder blend was then granulated by adding the granulation liquid consisting of methacrylic acid—methyl methacrylate copolymer (1:1) in ethanol whilst mixing for approximately 6 minutes (in the range 5-10 minutes) in a high shear granulator.

Following additional wet mixing (for approximately 15 seconds) the wet mass was milled in a Glatt rotating impeller mill. The wet mass was then dried in a hot air oven or a fluid bed drier and the dried mass was milled in a hammer conventional mill.

The granules were finally mixed in a blender with sodium stearyl fumarate, which was charged through a suitable sieve, and the granules compressed into tablets using a tablet press equipped with convex punches. A suitable tablet-coating was then applied using standard techniques.

### Example 7

### Extended Release Tablet

| | |
|---|---|
| Compound A besylate | 198.0 mg |
| Hypromellose 50 mPas | 97.2 mg |
| Cellulose, microcrystalline | 18.0 mg |
| Cellulose, microcrystalline (course) | 18.0 mg |
| Methacrylic acid - methyl methacrylate copolymer (1:1) | 25.2 mg |
| Ethanol, anhydrous (removed during processing) | q.s. |
| Sodium stearyl fumarate | 3.6 mg |

Prepared using an analagous procedure to that described in Example 6 with the microcrystalline cellulose (course) added in the final mixing step.

### Example 8

### Extended Release Tablet

| | |
|---|---|
| Compound A besylate | 198.0 mg |
| Hypromellose 50 mPas | 104.4 mg |
| Cellulose, microcrystalline | 18.0 mg |
| Hydroxypropyl cellulose | 10.8 mg |
| Methacrylic acid - methyl methacrylate copolymer (1:1) | 25.2 mg |
| Ethanol, anhydrous (removed during processing) | q.s. |
| Sodium stearyl fumarate | 3.6 mg |

Prepared using an analagous procedure to that described in Example 6, but the methacrylic acid—methyl methacrylate copolymer (1:1) was charged in the dry mixing step. HPC in ethanol was then added and the mixture granulated.

In Examples 6 to 8, the sodium stearyl fumarate in the tablet core can be varied between 0.7 mg and 7.2 mg.

In Examples 6 to 8, a suitable tablet coating consists of Hypromellose 6 cPs (10.8 mg); Macrogols (2.7 mg); Titanium dioxide (1.6 mg); Colour iron oxide yellow, CI 77492 (0.32 mg) and water (q.s.)—water is removed during processing.

In Examples comprising Hypromellose (HPMC) and methacrylic acid—methyl methacrylate copolymer (1:1), the microcrystalline cellulose may be varied in a range of 5 to 15 wt. % of the formulation, or microcrystalline cellulose may be included together with hydroxypropyl cellulose (HPC) in a combined range of 5 to 20 wt. % of the formulation. Additional mannitol may also be included in a range of 5 to 10 wt. % of the formulation. If HPC is included, this may be added in a granulation liquid in ethanol and the methacrylic acid—methyl methacrylate copolymer (1:1) added in a dry mixing step (see Example 8).

### Example 9

### Extended Release Tablet

| | |
|---|---|
| Compound A | 150.0 mg |
| Hypromellose K100 | 87.0 mg |
| Cellulose, microcrystalline | 30.0 mg |
| Methacrylic acid - methyl methacrylate copolymer (1:1) | 30.0 mg |
| Ethanol, anhydrous (removed during processing) | q.s. |
| Sodium stearyl fumarate | 3.0 mg |

Compound A in crystalline form is prepared according to the information contained in WO 2008/068475.

Hypromellose, microcrystalline cellulose and crystalline Compound A were blended. The powder blend was granulated by adding the granulation liquid consisting of methacrylic acid—methyl methacrylate copolymer (1:1) in ethanol while mixing, followed by additional wet mixing. The wet mass was milled in a suitable mill.

The wet mass was dried in a hot air oven or a fluid bed drier and the dried mass was milled in a suitable mill. The granules were final mixed with sodium stearyl fumarate, which was charged through a suitable sieve. The granules were compressed into tablets using a tablet press equipped with convex punches.

1. An extended release pharmaceutical formulation comprising compound Ph(3-Cl)(5-OCHF₂)—(R)CH(OH)C(O)—(S)Aze-Pab(OMe), or a pharmaceutically acceptable salt thereof, and a pharmaceutically acceptable diluent or carrier.

2. The extended release pharmaceutical formulation according to claim 1 wherein drug-drug interactions are limited between the compound Ph(3-Cl)(5-OCHF₂)—(R)CH(OH)C(O)—(S)Aze-Pab(OMe), or a pharmaceutically acceptable salt thereof, and other concomitantly dosed drug/s which are metabolised by CYP-450 enzymes.

3. The extended release pharmaceutical formulation according to claim 2 wherein the other concomitantly dosed drug/s is/are metabolised by CYP-450 isoenzyme 3A.

4. The extended release pharmaceutical formulation according to claim 1, wherein the pharmaceutically acceptable salt of Ph(3-Cl)(5-OCHF₂)—(R)CH(OH)C(O)—(S)Aze-Pab(OMe) is a sulfonic acid salt.

5. The extended release pharmaceutical formulation according to claim 4, wherein the pharmaceutically acceptable salt of Ph(3-Cl)(5-OCHF₂)—(R)CH(OH)C(O)—(S)Aze-Pab(OMe) is the benzenesulfonic acid salt.

6. The extended release pharmaceutical formulation according to claim 5, wherein the pharmaceutically acceptable salt of Ph(3-Cl)(5-OCHF₂)—(R)CH(OH)C(O)—(S)Aze-Pab(OMe) is the benzenesulfonic acid salt characterised by an X-ray powder diffraction pattern characterised by peaks with d-values at 5.9, 4.73, 4.09 and 4.08 Å.

7. The extended release pharmaceutical formulation according to claim 1 wherein the formulation comprises a gelling matrix.

8. The extended release pharmaceutical formulation according to claim 7 wherein the matrix comprises HPMC.

9. The extended release pharmaceutical formulation according to claim 7 wherein the matrix comprises methacrylic acid.

10. The extended release pharmaceutical formulation according to claim 1 wherein the formulation is a pellet formulation which comprises two coating layers.

11. The extended release pharmaceutical pellet formulation according to claim 10 wherein the formulation comprises an inner enteric coat.

12. The extended release pharmaceutical pellet formulation according to claim 10 wherein the formulation comprises an inner coat and an outer layer.

13. (canceled)

14. A method of treating a cardiovascular disorder in a patient suffering from, or at risk of, said disorder, while limiting drug-drug interactions with other concomitantly dosed drugs, comprising administering to the patient a therapeutically effective amount of an extended release pharmaceutical formulation according to claim 1.
